(19) United States
(12) Patent Application Publication
(10) Pub. No.: US 2025/0205416 A1
(43) Pub. Date: Jun. 26, 2025

(54) INFUSION THERAPY DEVICE WITH OCCLUSION DETECTION

(71) Applicants: BAXTER INTERNATIONAL INC., Deerfield, IL (US); BAXTER HEALTHCARE SA, Glattpark (Opfikon) (CH)

(72) Inventors: Mark Dhillon, San Diego, CA (US); Rachel Ping, Waukegan, IL (US); James Knapp, Crystal Lake, IL (US)

(21) Appl. No.: 18/984,621

(22) Filed: Dec. 17, 2024

Related U.S. Application Data
(60) Provisional application No. 63/612,762, filed on Dec. 20, 2023.

(51) Int. Cl. A61M 5/142; A61M 5/168; G16H 40/63

(52) U.S. Cl. CPC A61M 5/142; A61M 5/168; G16H 40/63; A61M 2005/16863; A61M 2202/0007; A61M 2202/04; A61M 2205/10; A61M 2205/18; A61M 2205/3334; A61M 2205/50; A61M 2205/583

(57) ABSTRACT

An infusion pump for detecting an occlusion is provided. The memory stores instructions that cause the one or more processors to input data into a trained neural network, and generate an alert when the trained neural network outputs an amount of occlusion flags above a predetermined threshold.

### RELATED APPLICATIONS

This application claims priority to U.S. Provisional Patent Application No. 63/612,762 filed Dec. 20, 2023, entitled “INFUSION THERAPY DEVICE WITH OCCLUSION DETECTION”, the entire contents of which is hereby incorporated by reference and relied upon.

### BACKGROUND

Generally, medical patients sometimes require precise intravenous (“IV”) delivery of either continuous medication or medication at set periodic intervals. Known infusion pumps provide controlled drug infusion therapy where the drug can be administered at a precise rate that keeps the drug concentration within a therapeutic margin and out of an unnecessary or possibly toxic range. Basically, infusion pumps provide appropriate drug delivery to the patient at a controllable rate, which does not require frequent attention.

Infusion pumps may facilitate administration of IV therapy to patients both in and outside of a clinical setting. Outside a clinical setting, doctors have found that in many instances patients can return to substantially normal lives, provided that they receive periodic or continuous IV administration of medication. Among the types of therapies requiring this kind of administration are antibiotic therapy, chemotherapy, pain control therapy, nutritional therapy, and several other types known by those skilled in the art. In many cases, patients receive multiple daily therapies. Certain medical conditions require infusion of drugs in solution over relatively short periods such as from thirty minutes to two hours. These conditions and others have combined to promote the development of increasingly lightweight, portable, or ambulatory infusion pumps that are configured to be worn by a patient and are capable of administering a continuous supply of medication at a desired rate, or provide several doses of medication at scheduled intervals.

Known infusion pumps include elastomeric pumps, which squeeze solution from flexible containers, such as balloons, into IV tubing for delivery to a patient. Alternatively, infusion pumps may include spring-loaded pumps that pressurize solution containers or reservoirs. Certain infusion pump designs utilize cartridges containing flexible compartments that are squeezed by pressure rollers for discharging the solutions. Further, known infusion pumps include peristaltic pumps having finger actuators or a roller actuator that apply pressure to IV tubing for delivering fluid from a fluid container to a patient. A syringe pump is another common type of infusion pump. Generally, a syringe pump includes a housing, which accepts a syringe assembly, and a drive mechanism for actuating the syringe plunger, thereby infusion liquid into the patient.

In order to ensure a safe and effective operation of infusion therapy, infusion pumps may need to be monitored for the risk of an occlusion. An occlusion involves any blockage or a closing of IV tubing connected to an infusion pump, which may thereby restrict the flow of medication to a patient. An occlusion can occur for a variety of reasons, including, but not limited to, unintended pressure applied to an infusion pump, a bending of IV tubing, and clogging caused by particles within a fluid.

Conventional methods for occlusion detection use a force sensor to detect an in-line IV tubing pressure. These force sensor measurements indirectly indicate an occlusion when the in-line pressure reaches or exceeds a threshold. For example, IV tubing may be placed into a tube channel of an infusion pump. The IV tubing may be pressed up against a force sensor using a cover of the infusion pump. This compressive force from the cover may be similar in magnitude to a pressure caused by an occlusion, which may cause the force sensor to transmit force measurements that are incorrectly interpreted as an occlusion being present. The force applied by the cover must therefore be removed from the force sensor output to properly assess the fluid pressure within the IV tubing. However, external factors, such as a height of the IV connection to a patient and characteristics of various devices associated with the infusion pump (e.g., a fluid container, a pumping chamber, the IV tubing, etc.) can also influence measurements by the force sensor, thereby affecting the reliability of using the force sensor to determine an occlusion. In addition, the IV tubing that was originally pressed up against the force sensor may gradually relax over time within the confined tube channel of the infusion pump. This can reduce the pressure measured by the force sensor, which further affects the reliability of this approach for occlusion detection.

Accordingly, more accurate and reliable methods and systems for detecting occlusions in infusion pumps are desired.

### SUMMARY

The present invention is generally directed to an infusion pump for delivering a flowable material, such as a fluid medication, to a patient through an infusion therapy line having occlusion detection.

In light of the disclosure herein and without limiting the disclosure in any way, in a first aspect of the present disclosure, which may be combined with any other aspect listed herein unless specified otherwise, an infusion pump for detecting an occlusion includes a pumping mechanism operable with a portion of intravenous (“IV”) tubing for providing controlled delivery of a fluid from a container to a patient, one or more processors, and a memory storing instructions. When executed by the one or more processors, the instructions cause the one or more processors to input data at predetermined intervals during an infusion session into a trained neural network, wherein the trained neural network outputs an occlusion flag or a non-occlusion flag, and generate an alert when the trained neural network outputs an amount of occlusion flags above a predetermined threshold.

In accordance with a second aspect of the present disclosure, which may be used in combination with any other aspect listed herein unless stated otherwise, the trained neural network includes an input layer, a hidden layer, and an output layer.

In accordance with a third aspect of the present disclosure, which may be used in combination with any other aspect listed herein unless stated otherwise, the output layer of the trained neural network outputs the occlusion flag or the non-occlusion flag.

In accordance with a fourth aspect of the present disclosure, which may be used in combination with any other aspect listed herein unless stated otherwise, the data is a three-dimensional dataset including a plurality of input vectors. Each one of the plurality of input vectors corresponds to a plurality of infusion pump parameters.

In accordance with a fifth aspect of the present disclosure, which may be used in combination with any other aspect listed herein unless stated otherwise, the plurality of infusion pump parameters comprises an ADC value, a flow rate, and a syringe size.

In accordance with a sixth aspect of the present disclosure, which may be used in combination with any other aspect listed herein unless stated otherwise, the data is an ADC value.

In accordance with a seventh aspect of the present disclosure, which may be used in combination with any other aspect listed herein unless stated otherwise, the trained neural network derives a plurality of infusion pump parameters from the ADC value.

In accordance with an eighth aspect of the present disclosure, which may be used in combination with any other aspect listed herein unless stated otherwise, the predetermined threshold is forty occlusion flags.

In accordance with a ninth aspect of the present disclosure, which may be used in combination with any other aspect listed herein unless stated otherwise, the trained neural network comprises a Convolutional Neural Network (“CNN”).

In accordance with a tenth aspect of the present disclosure, which may be used in combination with any other aspect listed herein unless stated otherwise, the CNN comprises a Residual Network (“ResNet”) Architecture.

In accordance with an eleventh aspect of the present disclosure, which may be used in combination with any other aspect listed herein unless stated otherwise, the one or more processors are configured to cause an alert or an alarm to be displayed on a user interface when the amount of occlusion flags exceed the predetermined threshold.

In accordance with a twelfth aspect of the present disclosure, which may be used in combination with any other aspect listed herein unless stated otherwise, the one or more processors are configured to pause or terminate the infusion session when the amount of occlusion flags exceed the predetermined threshold.

In accordance with a thirteenth aspect of the present disclosure, which may be used in combination with any other aspect listed herein unless stated otherwise, a method of training a neural network for detecting an occlusion in an infusion pump includes collecting a plurality of infusion pump parameters, correlating the plurality of infusion pump parameters to an occlusion state of the infusion pump, and inputting the plurality of infusion pump parameters correlated to the occlusion state into the neural network. The occlusion state corresponds to a time at which the plurality of infusion pump parameters was collected.

In accordance with a fourteenth aspect of the present disclosure, which may be used in combination with any other aspect listed herein unless stated otherwise, the plurality of infusion pump parameters is collected from a plurality of infusion pumps.

In accordance with a fifteenth aspect of the present disclosure, which may be used in combination with any other aspect listed herein unless stated otherwise, the occlusion state represents an occlusion or no occlusion.

In accordance with a sixteenth aspect of the present disclosure, which may be used in combination with any other aspect listed herein unless stated otherwise, the neural network comprises a Convolutional Neural Network (“CNN”).

In accordance with a seventeenth aspect of the present disclosure, which may be used in combination with any other aspect listed herein unless stated otherwise, the CNN comprises a Residual Network (“ResNet”) Architecture.

In accordance with an eighteenth aspect of the present disclosure, which may be used in combination with any other aspect listed herein unless stated otherwise, an infusion pump method for detecting an occlusion includes determining a real time ADC value, applying the real time ADC value to a trained neural network, and generating an occlusion alarm when a number of occlusion flags exceed a threshold. The trained neural network outputs a Boolean determination corresponding to an occlusion flag.

In accordance with a nineteenth aspect of the present disclosure, which may be used in combination with any other aspect listed herein unless stated otherwise, the trained neural network derives a plurality of infusion pump parameters from the real time ADC value.

In accordance with a twentieth aspect of the present disclosure, which may be used in combination with any other aspect listed herein unless stated otherwise, the one or more processors are configured to cause an alert or an alarm to be displayed on a user interface when the amount of occlusion flags exceed a predetermined threshold.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIGS. 1A and 1B are isometric views of a syringe pump, according to an example embodiment of the present disclosure.

FIGS. 2A, 2B, 2C, and 2D are perspective views of a syringe pump, according to an example embodiment of the present disclosure.

FIGS. 3A and 3B are partial views of a syringe compartment of a syringe pump, according to an example embodiment of the present disclosure.

FIG. 4 is a partial view of a flange plate of a syringe pump, according to an example embodiment of the present disclosure.

FIGS. 5A and 5B are isometric views of a drive head of a syringe pump, according to an example embodiment of the present disclosure.

FIG. 5C is an exploded isometric view of a drive head of a syringe pump, according to an example embodiment of the present disclosure.

FIGS. 6A and 6B are cross-sectional views of drive mechanism components of a syringe pump, according to an example embodiment of the present disclosure.

FIGS. 7A and 7B are perspective views of a peristaltic pump with a door in a closed position, according to an example embodiment of the present disclosure.

FIGS. 7C and 7D are perspective views of a peristaltic pump with a door in an open position, according to an example embodiment of the present disclosure.

FIG. 8 is an example matrix showing an occlusion training dataset, according to an embodiment of the present disclosure.

FIG. 9 is an example vector showing target values for the example occlusion training dataset of FIG. 8, according to an example embodiment of the present disclosure.

FIG. 10 is a flow chart illustrating a method for training a neural network for occlusion detection, according to an embodiment of the present disclosure.

FIG. 11 is a flow chart illustrating a method for occlusion detection in infusion pumps using a trained neural network, according to an embodiment of the present disclosure.

FIGS. 12A and 12B are example vectors showing an ADC value that corresponds to infusion pump parameters, according to an embodiment of the present disclosure.

FIGS. 13A and 13B are high-level component diagrams of an infusion pump, according to an example embodiment of the present disclosure.

### DETAILED DESCRIPTION OF EXAMPLE EMBODIMENTS

In-line occlusion detection is critical for the safe and effective operation of infusion pumps. The most common method for occlusion detection utilizes a force sensor to detect in-line pressure (e.g., a pressure caused by fluid within an IV tubing coupled to an infusion pump). The detected in-line pressure can indirectly indicate an occlusion status (e.g., a presence or an absence of an occlusion) based on whether or not the detected in-line pressure reaches or exceeds a preset threshold. However, false alarms of occlusion can occur for a variety of factors, including, but not limited to, variations in infusion pumps and variations regarding the patient. The present disclosure provides more robust, accurate, and reliable systems and methods for occlusion detection in infusion pumps. Furthermore, the present disclosure includes novel and nonobvious systems and methods for using artificial intelligence models specifically suited to detect upstream and downstream occlusions, which take place in the infusion pumps.

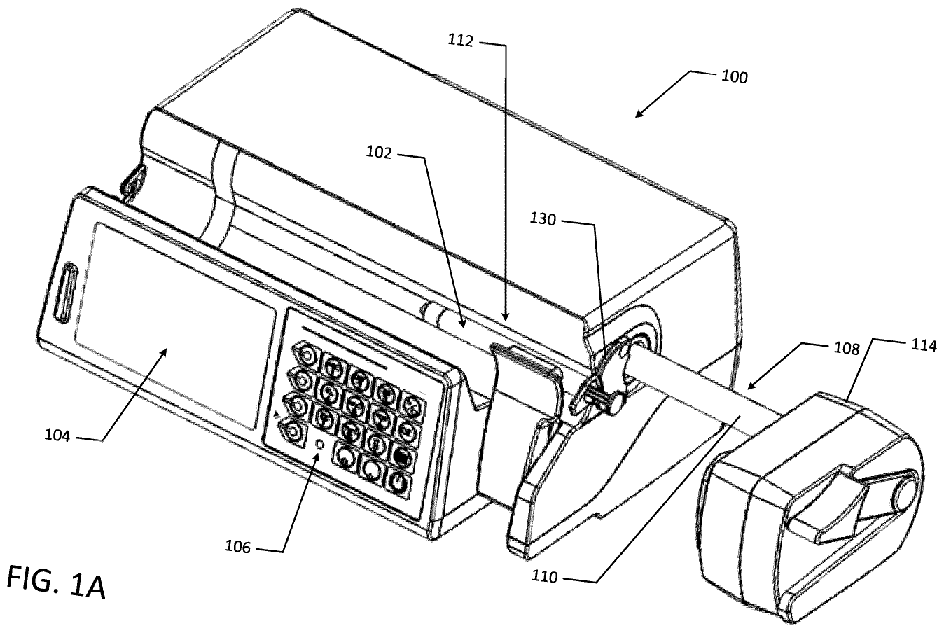

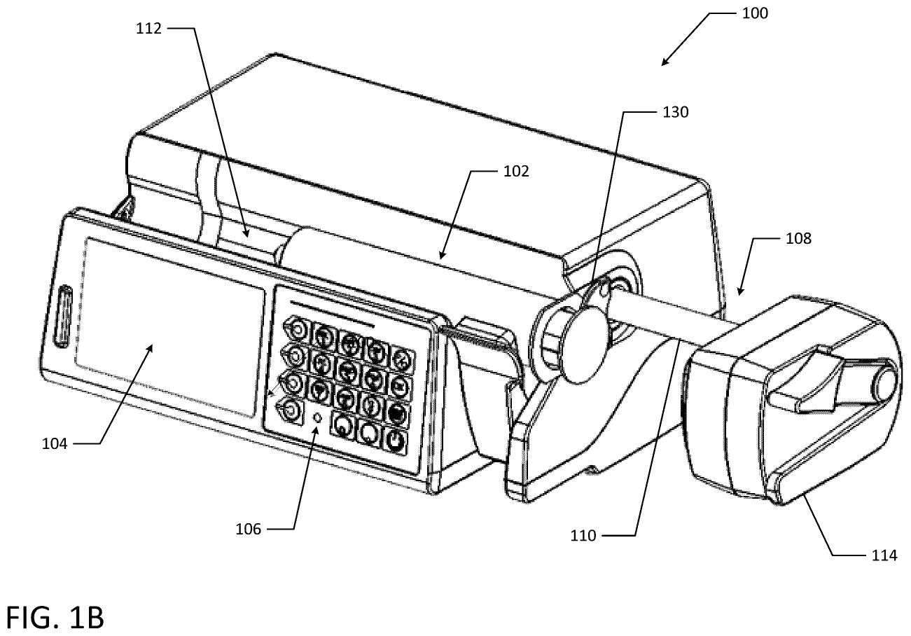

FIGS. 1A and 1B are isometric views of a syringe pump, according to an example embodiment of the present disclosure. The syringe pump 100 includes a housing that supports a syringe assembly 102, a user interface or a display 104, a keypad 106, a power supply, a drive head 114, and a drive mechanism 108. The drive mechanism 108 includes a lead screw, a split-nut, and a drive rod 110. As discussed in more detail below, the syringe pump 100 may also include a syringe sensor system. Other examples of syringe pumps include the syringe pump as described in U.S. Pat. No. 7,608,060, the entirety of which is incorporated herein by reference. The above example is non-limiting and the concepts disclosed herein could apply to other medical devices and/or syringe pumps.

The housing houses various components of the syringe pump 100, including the user interface or display 104 that includes the display screen and a keypad 106. At a bottom, front portion of the housing, a syringe compartment 112 is defined. The syringe compartment 112 is configured to accommodate the syringe assembly 102. The housing can be made from a variety of materials including various types of plastics and metals.

The user interface or display 104 generally includes a display screen. The display screen may act as a touch screen for a user to input data into the syringe pump 100. The user interface or display 104 and the keypad 106 are used to program the syringe pump 100, and more specifically, a processor in the syringe pump 100 to set the fluid delivery amount, etc., which is later communicated to the drive mechanism 108. The syringe pump 100 and user interface or display 104 may utilize additional identification features regarding the medication delivered by the syringe pump 100. For example, the syringe pump 100 may be equipped with a radio frequency identification (“RFID”) reader or other identification reader that cooperates with an RFID tag or other identifier attached to a syringe barrel. The RFID tag is configured to store significant information including, but not limited to, the type of medication, amount, concentration, as well as parameters related to the syringe pump 100 and instructions for the medication.

The display screen may be equipped with a pad about the outer periphery of the screen. The pad is a shock absorbent member made preferably of an elastomeric material. In one preferred embodiment, the pad is made from polyurethane. The pad absorbs forces generated if the syringe pump 100 is jostled, bumped or dropped, and minimizes the effect such occurrences have on the display screen.

The syringe pump 100 includes a power supply that can take many different forms. In one preferred embodiment, the power supply may be in the form of a rechargeable battery unit. Additionally, the syringe pump 100 may be powered from an AC power supply. The AC power supply assembly has a power cord and an associated terminal that plugs into the housing. The AC power supply assembly has a plug that is configured to be inserted into a standard electrical outlet to recharge the rechargeable battery when necessary. The AC power can also be supplied through the assembly to power the syringe pump 100.

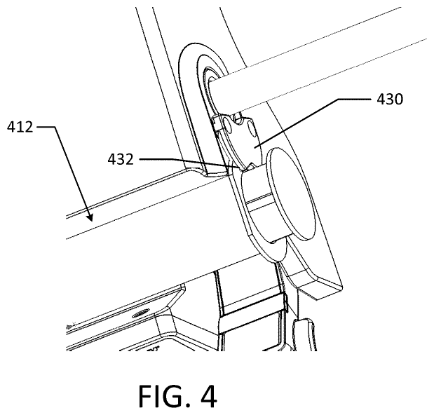

As introduced above, the syringe compartment 112 is generally dimensioned to receive and support the syringe assembly 102. The syringe assembly 102 includes a syringe barrel, a syringe plunger, and a syringe barrel flange. The syringe barrel contains medication and slidably receives the syringe plunger. The syringe plunger is driven by the drive mechanism 108 to force medication from the syringe barrel through a tube (not shown) to a patient. The tube would have one end connected to an end of the syringe barrel and another end adapted to be connected to a patient. The syringe barrel flange engages a flange plate 130 of the syringe pump 100. The flange plate 130 holds the syringe barrel flange against the housing of the syringe pump 100 under a compressive force. Thus, the flange plate 130 secures the syringe assembly 102 to the syringe pump 100. In an example, the flange plate 130 is spring biased towards the syringe pump housing. The flange plate 130 is further illustrated and described in reference to FIG. 4.

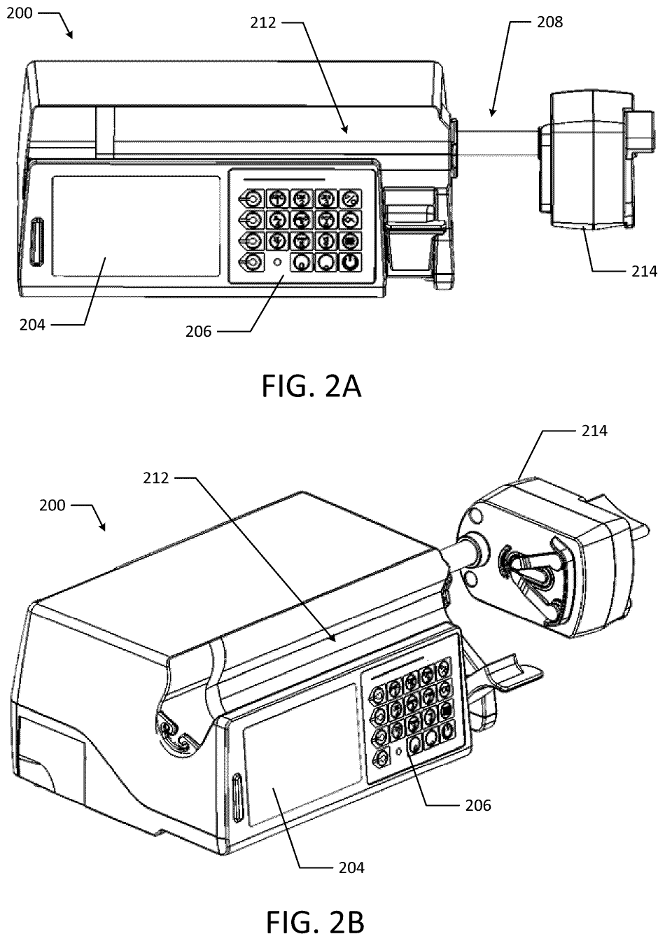

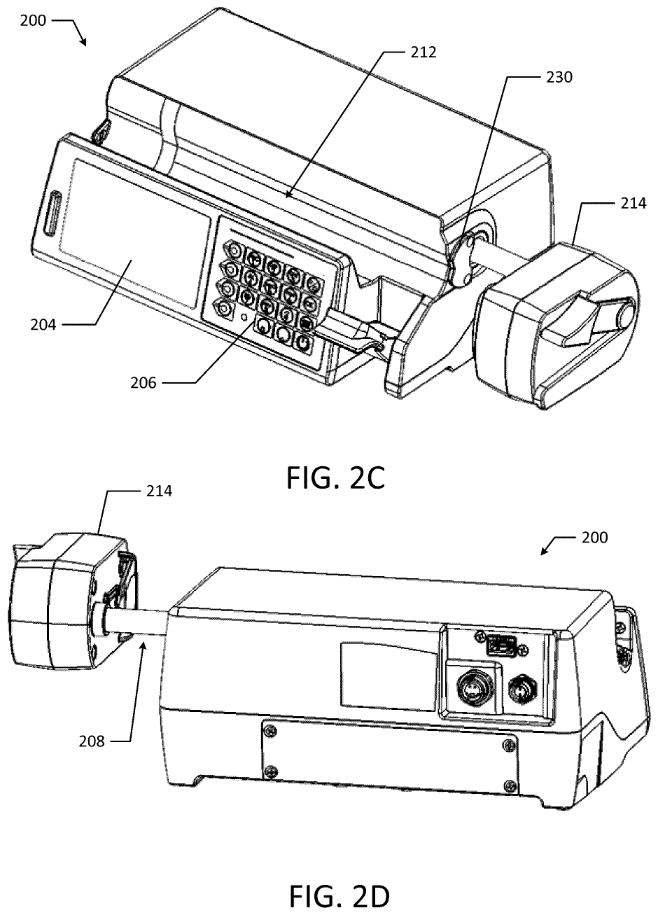

FIGS. 2A, 2B, 2C, and 2D are perspective views of a syringe pump, according to an example embodiment of the present disclosure. Similar to FIGS. 1A and 1B, the syringe pump 200 illustrated in FIGS. 2A to 2D generally includes a user interface or display 204, a keypad 206, a power supply, a drive mechanism 208, a drive head 214, and a flange plate 230. The housing houses various components of the syringe pump 200 including the user interface or display 204 that includes a display screen and a keypad 206. At a bottom, front portion of the housing, a container compartment or syringe compartment 212 is defined that accommodates the syringe assembly.

### Syringe Barrel Position

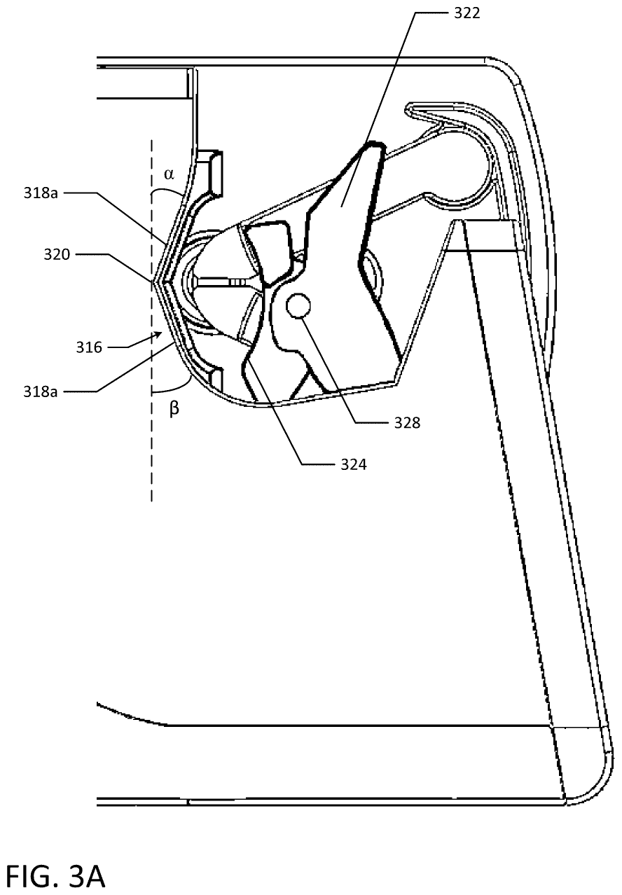

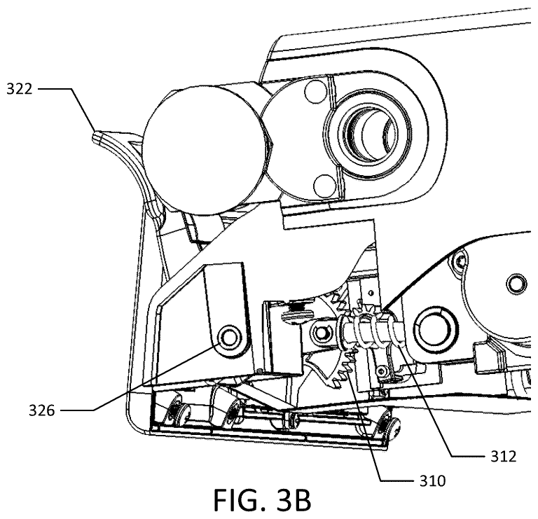

FIGS. 3A and 3B are partial views of a syringe compartment of a syringe pump, according to an example embodiment of the present disclosure. As illustrated in FIGS. 3A and 3B, the syringe compartment has a rear wall 316 that is generally concave to receive the syringe barrel of the syringe assembly (not shown). The syringe barrel of the syringe assembly and the rear wall 316 are generally in confronting relation. The rear wall 316 may include a top portion 318a and a bottom portion 318b that meet at a vertex 320. The top portion 318a has a wall angle (α) with respect to a vertical plane that intersects the vertex 320. Similarly, the bottom portion 318b has a wall angle (β) with respect to a vertical plane that intersects the vertex 320, for example 20 degrees, which improves consistent syringe barrel position and assists with securing the syringe assembly. The wall angles (α, β) may be the same or different. For example, the wall angle (α) for the top portion 318a may be larger than the wall angle (β). In another example, the wall angle (α) for the top portion 318a may be smaller than the wall angle (β) for the bottom portion 318b. The larger the wall angle (α, β), the larger distance between the centers of the smallest and largest syringe assemblies loaded in the syringe pump. For example, a flat rear wall 316 would allow the center of a syringe barrel to sit at the syringe barrel radius from the rear wall 316. If the rear wall is convex, the center of a syringe barrel is moved further and further away from the vertex 320 as the syringe barrel diameter increases. For example, a small diameter syringe assembly can be positioned close to the vertex 320, but a large diameter syringe assembly may contact the top and bottom portions, 318a and 318b, of the rear wall 316 at a greater distance from the vertex 320. Even though larger wall angles (α, β) create larger distances between the centers of different syringe assemblies loaded in the syringe pump, larger wall angles (α, β) advantageously help mitigate syringe assembly misloading by providing a deeper notch to guide the syringe assembly towards the vertex 320 of the rear wall 316.

### Syringe Barrel Clamp

As illustrated in FIGS. 3A and 3B, the syringe pump may further comprise a syringe barrel clamp 322 that is movably mounted in the syringe compartment. The syringe barrel clamp 322 has a concave inner surface 324 that faces the rear wall 316 and that fits over the syringe barrel. The syringe barrel clamp 322 is pivotable to move towards and away from the rear wall 316. Additionally, the syringe barrel clamp 322 is pivotable to accommodate different sized syringe barrels. The syringe barrel clamp 322 is configured to pivot towards the rear wall 316 or a home closed position as well as away from the rear wall 316 to an open position. For example, as illustrated in FIG. 3B, the syringe barrel clamp 322 may be spring biased to pivot about pivot 326 to find a home closed position or consistent position such that the syringe barrel clamp 322 is adapted to distinguish different syringe assemblies as well as consistently place barrels of the same size. The spring bias towards the home closed position also ensures that a syringe assembly loaded into the syringe pump does not come loose if the syringe pump is accidentally bumped. As the syringe barrel clamp 322 is opened, after a certain point in rotation, the syringe barrel clamp 322 is biased towards an open position instead of the home closed position. For example, as a user is opening the syringe barrel clamp 322, after a certain degree of rotation, the syringe barrel clamp 322 becomes biased to the open position, which advantageously facilitates ease of loading a syringe assembly (e.g., one handed syringe loading). As the user begins to close the syringe barrel clamp 322, the syringe barrel clamp 322 rotates beyond the open position bias and again becomes biased towards the home closed position.

The concave inner surface 324 of the syringe barrel clamp 322 is included on a swivel 328 to aid in retaining different size syringe assemblies. Both the swivel 328 and the pivot 326 are adapted to consistently locate and position syringe assemblies in the syringe pump. The pivot 326 controls the point about which the entire syringe barrel clamp 322 rotates while the swivel 328 controls the orientation of the barrel-contacting surface of the syringe barrel clamp 322. The syringe pump may be compatible with and support various syringe sizes (e.g., 1 mL, 3 mL, 5 mL, 10 mL, 20 mL, 30 mL, 50 mL, and 60 mL syringes) from various syringe manufacturers. Additionally, the syringe pump housing may nest the syringe barrel clamp 322, protecting the syringe barrel clamp 322 from accidental bumps or unintentional clamp engagement from either side of the housing.

In an example, the syringe barrel loading includes barrel size detection means. For example, a rotary potentiometer may be used to detect the size of a syringe barrel. As illustrated in FIG. 3B, rotation of the syringe barrel clamp 322 may be detected based on an amount of rotation or travel of gears 310 and/or compression of a spring 312. Further, the rotary potentiometer may be geared up 3:1 to obtain appropriate resolution to differentiate between syringe sizes. In another example, a linear potentiometer may be used to detect the size of the syringe barrel.

### Syringe Barrel Flange Clamp

FIG. 4 is a partial view of a flange plate of a syringe pump, according to an example embodiment of the present disclosure. As introduced above, the syringe assembly 412 is secured to the syringe pump by the flange plate 430. The flange plate 430 includes an angled profile 432 to assist the syringe barrel flange to slide into place. For example, the angled profile 432 allows a syringe barrel flange to be inserted under the outer lip of flange plate 430 and, as the syringe barrel flange is moved further towards the syringe pump housing, the angled profile 432 guides the syringe barrel flange in towards the syringe pump housing and under the flange plate 430 until the force overcomes the spring bias. Once a user overcomes the retention force from the spring bias, the flange plate 430 extends away from the syringe pump housing to accommodate the width of the syringe barrel flange. The spring bias retains the syringe barrel flange against the syringe pump housing under a compressive force until the syringe is later removed by a user.

### Syringe Flippers

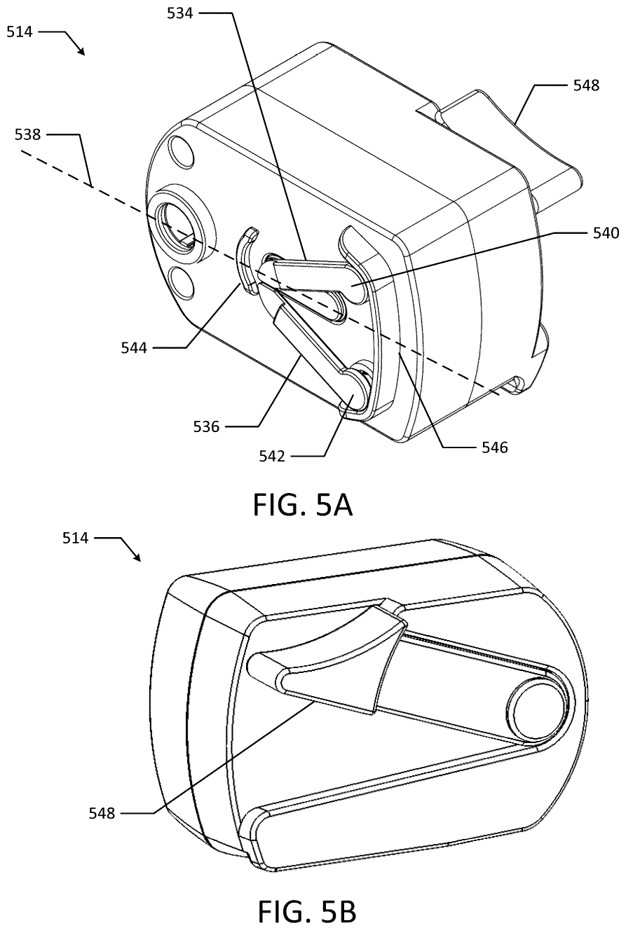

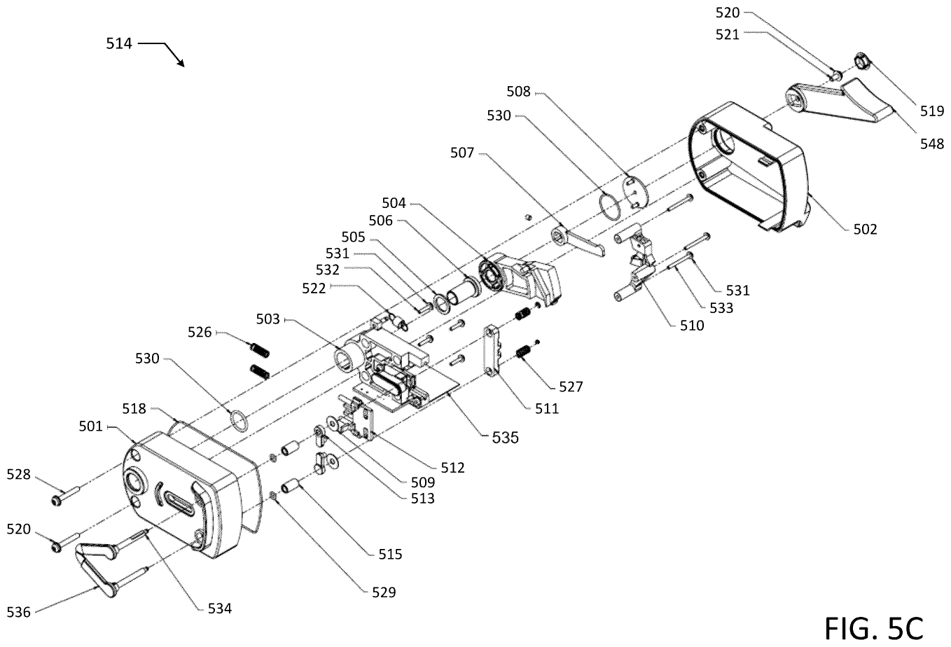

FIGS. 5A and 5B are isometric views of a drive head of a syringe pump, according to an example embodiment of the present disclosure. The drive head 514 of the drive mechanism includes two syringe flippers or plunger hooks 534 and 536 on the plunger driver. The size and orientation of the plunger hooks 534, 536 allow full compression of the syringe plunger. The plunger hooks 534, 536 are biased towards a syringe plane 538 about pivots 540 and 542. The pivots 540, 542, positioned on the distal-most side of the drive head 514 away from the syringe pump housing, reduce plunger hook interference with the syringe pump to allow full compression of the syringe plunger.

In an example, a syringe retaining wall 544 may be included on the plunger drive head 514 to help position and retain the plunger thumb flange on the drive head 514. Additionally, an outside wall 546 may be included on the plunger driver to protect the plunger during infusion therapy. As illustrated in FIG. 5B, the drive head 514 also includes a plunger lever 548. The plunger lever 548 includes a thumb compression region, which provides an intuitive compression feature on the lever to assist user interaction. Actuation of plunger lever 548 opens plunger hooks 534, 536 (e.g., plunger hooks 534, 536 away from each other and syringe plane 538) allowing a user to position a syringe in a syringe pump. After releasing plunger lever 548, the plunger hooks pivot back towards syringe plane 538 and hold the plunger stem and thumb flange of the syringe.

Actuation of the plunger lever 548 may also move plunger hooks 534, 536 out from the drive head 514 towards the syringe barrel to provide additional space between the plunger hooks 534, 536 and the pushing face of the drive head 514. The plunger hooks 534, 536 may be spring biased toward the pushing face of the drive head 514 to accommodate a wide variety of plunger flange thicknesses. The bias towards the drive head 514 and the syringe plane 538 enable the plunger hooks 534, 536 to self-adjust to the size of the syringe plunger mounted in the syringe pump.

In an example, the plunger hooks 534, 536 are spring loaded and biased inward to provide a substantial clamping force against the stem of the syringe plunger as well as a compressive force that holds the plunger thumb flange against the drive head 514. The plunger hooks 534, 536 may first pivotally close before moving laterally towards the drive head 514 and returning toward their lateral home position. This sequence of closing motions ensures that the plunger stem is first centered by the arms, and then plunger thumb flange is brought into contact with the pushing surface.

FIG. 5C is an exploded isometric view of a drive head of a syringe pump, according to an example embodiment of the present disclosure. The drive head 514 may include a housing having a front case 501 and a rear case 502, which may have a gasketed interface (e.g., gasket 518) therebetween. The front case 501 and the rear case 502 may be coupled together via a snap-fit connection and may be secured via screws 528 with optional washers 520. The plunger lever 548 may be positioned on the rear case 502 and coupled via lever clasp 519, screw 521 and/or washer 520. The plunger lever 548 is operatively coupled to the drive rod via a plunger lever coupling 508, lever spring 522, clutch link 507, plunger hook lever 504, and plunger fix screw 506, which extends through the plunger base sensor assembly 503. The connections may also include O-rings 530 and washer 505.

Plunger hooks 534 and 536 are positioned on the front case 501 and extend through the housing and interface with hook slide plate 511, hook opener holder assembly 510, and hook opening plate assembly 512. The hook opening plate assembly 512 may include hook-opening springs 526 and may be coupled to a plunger hook sensor PCB which may include an SMT switch. The plunger hook sensor PCB may be mounted to the hook opening plate assembly 512 and the plunger base sensor assembly 503 via screws spaced by washers. The various mounting components for the plunger hooks 534 and 536 may include screws 533, washers 531, e-rings 521, springs 527, hook cover washers 509, hook links 513, plunger hook bushings 515, and plunger hook shaft O-rings 529.

The housing may house a plunger base sensor assembly 503 attached via washers and screws 532. The plunger base sensor assembly 503 may also include an insulation sheet 535. The plunger base sensor assembly 503 includes a plunger button that is held against a flat plastic plate within the plunger base sensor assembly 503, which directly contacts a cantilever beam inside the drive head 514. The cantilever beam may include a strain gauge to measure the force acting on the plunger button.

Rotation of the plunger lever 548 is directly translated to “in and out” movement of plunger hooks 534, 536. A wiper lever attached in the hook opener holder assembly 510 may compress the hook slide plate 511 to create the “in and out” movement of plunger hooks 534, 536. As the plunger hook lever 504 pushes and rotates the small wiper lever, the wiper's wedge feature interacts with a roller within the hook slide plate 511. The hook slide plate 511 is controlled by compressing springs or hook holding springs 527. As the roller tracks down the wedge, the springs 527 are compressed and the plunger hooks 534, 536 are pushed away from the front case 501.

Rotation of the plunger lever 548 is also directly translated to rotation of plunger hooks 534, 536 via the plunger lever coupling 508. For example, the plunger hook lever 504 and the plunger lever 548 are held in the upward rest position by an extension spring or lever spring 522. The lever spring 522 is stretched as the plunger lever 548 is compressed and then returns the system to the rest position or “lever up” position when the plunger lever 548 is released. Specifically, the plunger hook lever 504 interacts with a second roller, attached on the hook opening plate assembly 512 that follows a track within the plunger hook lever 504. As the roller follows the track, the entire hook opening plate assembly 512 compresses the hook opening springs 526 to move linearly. The plate within the hook opening plate assembly 512 may include two identical slots that fit within the hook link and create rotational movement. The hook links securely connect to the plunger hook upper assembly shafts, so the rotational motion created moves the plunger hooks 534, 536 open. When the user releases the plunger lever 548, the hook opening springs 526 return the hook opening plate assembly 512 back to its original position, which rotates the plunger hooks 534, 536 back to the closed position.

As discussed above, the plunger hooks 534, 536 allow full compression of the syringe plunger and avoid interference with flange plate thereby allowing the syringe pump to more fully expel fluid from the syringe. For example, if the plunger hooks 534, 536 were alternatively positioned in a mirrored orientation that pivoted on an opposite side of the housing (e.g., closer to drive rod), the hooks may interfere with flange plate preventing the syringe from fully compressing.

Each of the syringe loading components (e.g., barrel clamp, flange plate, flippers or plunger hooks) are adapted to capture and secure (e.g., load) a syringe in a manner that prevents an unintended bolus due to a syringe drive head impact. Discussed in more detail below, when a syringe is loaded horizontally with the barrel flush against the barrel support face or rear wall, the barrel flange in the flange groove provided by the flange plate, the barrel clamp pushing flush against the syringe barrel and the plunger thumb button within the plunger hooks, the syringe pump may detect the barrel flange presence, the plunger button presence, and may also be able to identify syringe barrel outside diameter dimensions.

### Syringe Pump Sensors

The syringe pump includes various sensors to ensure proper operation and syringe loading. For example, the syringe pump may include a clutch sensor, an occlusion sensor, a flange detection sensor, a barrel size sensor (e.g., barrel size measurement sensor), a syringe plunger position sensor, and a motor encoder. The flange detection sensor, barrel size sensor, and syringe plunger position sensor may work in conjunction as a syringe sensor system. As discussed above, the syringe barrel loading includes barrel size detection means, such as a barrel size sensor. The sensors may track plunger position. For example, the syringe plunger position sensor may include a linear pot wiper blade that is connected to the clutch and travels parallel with the lead screw. The resistance of the sensor in circuit changes directly with the travel and outputs the voltage as a function of linear position. In another example, the syringe plunger position sensor may be an electromagnetic sensor that includes a magnet and a plunger linear sensor array.

The output from the syringe plunger position sensor may be used to track the position of the syringe plunger. For example, tracked movements may be used to check if the syringe plunger movement matches programmed delivery rate. The syringe plunger position sensor may be used to check a Volume to Be Infused (“VTBI”) command with the available volume in the syringe pump. For example, the syringe plunger position may be used to determine the remaining length of the syringe barrel, which along with the barrel diameter may be used to determine the remaining volume in the syringe. The syringe plunger position sensor may also be used to detect if a syringe has emptied or if an empty syringe has been loaded (e.g., detects a fully dispensed plunger position). Additionally, the syringe plunger position sensor may be used to detect max deadstop (REOT, FEOT) position of the syringe plunger head as well as to detect slope between the motor and clutch.

The sensors may indicate syringe size. The syringe barrel size sensor may include a linear pot wiper blade connected to the shaft of the barrel clamp. The sensor resistance in the circuit changes proportional to the distance travel of the barrel clamp and outputs the voltage as a function of position. In another example, the syringe barrel size sensor may be an electromagnetic sensor that includes a magnet and a barrel linear sensor array. The magnet is mounted on the syringe barrel clamp assembly. The linear sensor array is mounted generally adjacent thereto and has a sensor. Because the movement of the syringe barrel clamp is less than the plunger movement, a single sensor can be used. Similar to the syringe plunger position sensor, based on the signal levels sensed by the sensor, the sensor is configured to determine what size syringe is loaded into the pump.

The syringe barrel size sensor may be used to indicate if an incompatible syringe is loaded in syringe pump. Additionally, the sensor may be used as a gross indication that a tube is not closed properly. In an embodiment, the flange detection sensor may be a micro switch that is located behind the barrel flange retention plate. In an example, the switch is depressed by the plate when the syringe flange is pushed into place during syringe loading. The flange detection sensor may indicate that the barrel is in its proper position and may be used to initiate pump power-up.

A syringe plunger button detect sensor positioned on the drive head detects the presence of a syringe. For example, a micro switch may be located in the plunger drive head such that the switch is depressed by the plunger flange when the syringe flippers or plunger hooks capture the plunger flange during syringe loading. The plunger button detect sensor indicates presence of the plunger flange at the drive head to verify the syringe is properly loaded.

A syringe force sensor or downstream occlusion (“DSO”) sensor may be located on the drive head of the syringe pump and may be used to indicate force or pressure on the syringe plunger. The calculated pressure may be used to determine a downstream occlusion, discussed in more detail below. Additionally, the force sensor may be used to perform start-up compensation to determine when the slack in syringe travel is removed. The syringe force sensor may also be used to detect a fully spent syringe as well as to detect the plunger flange.

The syringe pump, as previously described, has associated therewith a plurality of sensors, which are operative to provide information as to the function and location of the various elements thereof. A clutch sensor may comprise an optical switch with a mechanical shutter that moves to block light. The clutch sensor may indicate that the clutch is engaged or disengaged.

A motor position sensor may comprise a rotary magnetic encoder. For example, a magnet may be mounted on the motor shaft, which turns with the motor and actives the rotary encoder count, which indicates the position of the motor (e.g., 15 revolutions per 1 revolution of leadscrew per 1 mm linear travel of plunger). In another example, the syringe pump may include a drive motor shaft encoder comprising an encoder flag wheel attached to the armature shaft of the motor. The pump motor flag wheel may include a plurality of flags (e.g., twelve flags) extending radially outward from the hub thereof. The motor position sensor may indicate rotation of the motor and may be used to detect motor stall.

One or more of the above sensors (e.g., plunger position sensor) may be used to compensate for system slack during infusion therapy startup.

### Syringe Drive Mechanism

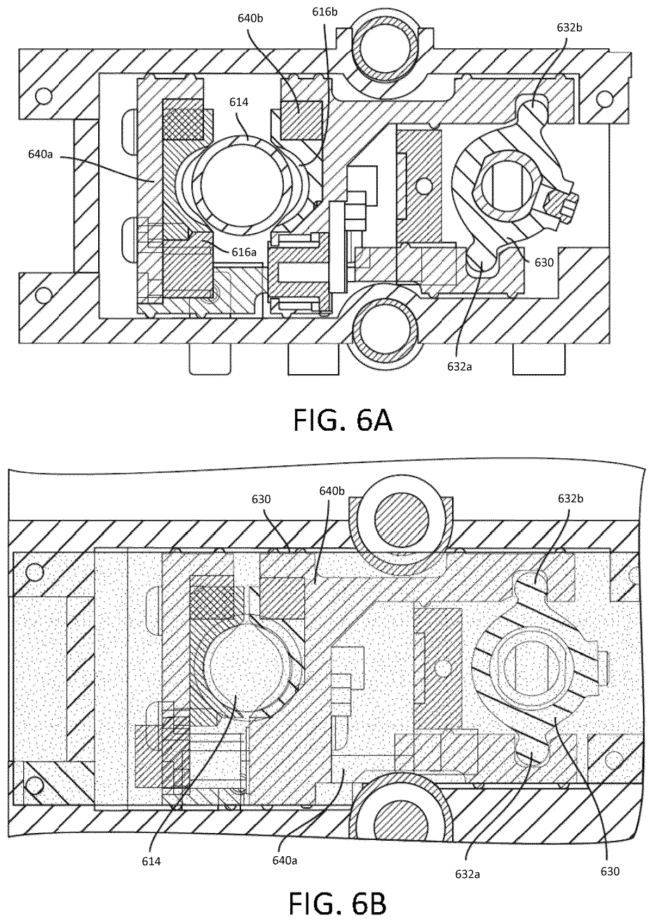

FIGS. 6A and 6B are cross-sectional views of drive mechanism components of a syringe pump, according to an example embodiment of the present disclosure. The syringe drive mechanism is accommodated by the syringe pump housing and generally includes a motor, a lead screw 614, a split-nut 616 (with nut halves 616a and 616b), a slide assembly, and a drive head. As illustrated in FIG. 6A, the split-nut 616 is in an unengaged state (e.g., not contacting lead screw 614) and in FIG. 6B, the split-nut 616 is in an engaged state (e.g., contacting lead screw 614).

The motor is operably connected to the lead screw 614 to rotate the lead screw 614 when the motor is energized. The lead screw 614 has threads that cooperate with a threaded member, such as the split-nut 616 of the slide assembly. The slide assembly is associated with the lead screw 614 and thus moves linearly in response to rotation of the lead screw 614 by the motor. Linear movement of the slide assembly and drive head moves the syringe plunger, having a plunger flange, a plunger arm, and plunger stopper, within the syringe barrel to expel fluid from the syringe assembly to a patient for infusion therapy.

Typical half-nut designs may include a clutch mechanism with a half-nut that is spring biased against the lead screw 614. However, the spring bias may lead to additional stress levels on the half-nut, which may ultimately lead to thread wear on the half-nut. The additional stress and wear may also contribute to periodic fluctuations within a lead screw rotation cycle. Additionally, during an occlusion, half-nut drive mechanisms may experience stress levels that exceed the half-nut's yield strength, which over time may lead to half-nut failure (e.g., the threads of the half-nut may be significantly to completely worn away leading to pump failure). The half-nut wear may release, for example, an abrasion from worn half-nut threads, which may interfere with other pump components.

The improved split-nut 616 disclosed herein provides over twice as much thread engagement/contact than with traditional half-nut designs. Additionally, the split-nut 616 allows threads of the nut to be more concentric, which helps lower the flow rate accuracy (“FRA”) periodic fluctuations and wear. For example, small movements of a half nut may create large variations of flow rate accuracy when threads are angled. Additionally, the halves 616a, 616b contact each other such that they are not biased against the lead screw 614, which advantageously lowers friction and wear and also increases reliability. An example lead screw material is SUM24L (Electroless Nickel Plating). An example split-nut material is C95400 Aluminum Bronze. The materials listed for the lead screw and split-nut are not limiting and are provided as an example. Any other suitable material may be used.

The split-nut 616 of the slide assembly is configured to be disengaged from the lead screw 614 allowing the slide assembly to freely slide along the lead screw 614 to linearly position the drive head against the syringe plunger extending from the syringe barrel. The nuts halves 616a, 616b are biased into engagement with the lead screw 614 by a spring and magnetic clutch. The threads on each of the nut halves 616a, 616b engage generally opposed sides of the lead screw 614. The split-nut configuration and anti-ratcheting clutch design maximizes performance and minimizes wear of the threads of the split-nut 616 and lead screw 614. With the threads engaged, when the motor rotates the lead screw 614, the split-nut 616 moves along the lead screw 614, which, in turn, linearly moves the drive head. This pushes the syringe plunger into the syringe barrel to displace medicament from the syringe assembly to the patient.

As mentioned above, the split-nut 616 can also be easily disengaged from the lead screw 614 which allows the slide assembly to be positioned along the lead screw 614 such as when positioning the drive head against the syringe plunger. As illustrated in FIGS. 6A and 6B, a toggle 630 may rotate to move a first frame or clutch member 640a and a second frame or clutch member 640b. For example, key 632a may engage a corresponding groove in clutch member 640a and key 632b may engage a corresponding groove in clutch member 640b. As the toggle 630 rotates counterclockwise (from FIG. 6A to 6B), the frame or clutch members 640a, 640b are simultaneously drawn towards lead screw 614. For example, the toggle 630 pulls clutch member 640a towards lead screw 614 and pushes clutch member 640b towards lead screw 614. As shown in FIG. 6B, when the toggle 630 is in a vertical orientation, the split-nut 616 is fully engaged with lead screw 614. To disengage the split-nut 616 from the lead screw 614, toggle 630 may be rotated clockwise to push clutch member 640a away from lead screw 614 and pull clutch member 640b away from lead screw 614.

### Peristaltic Pump

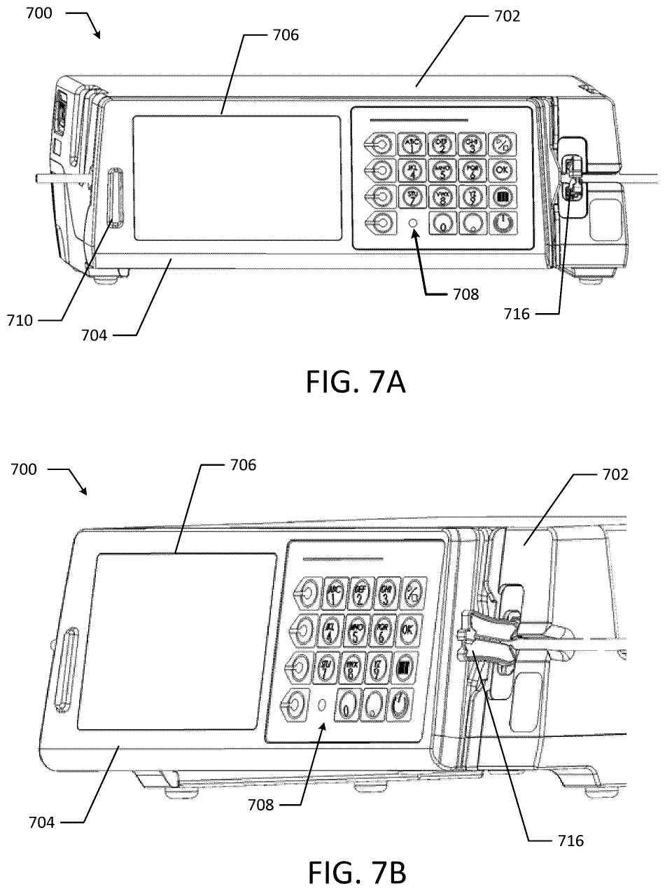

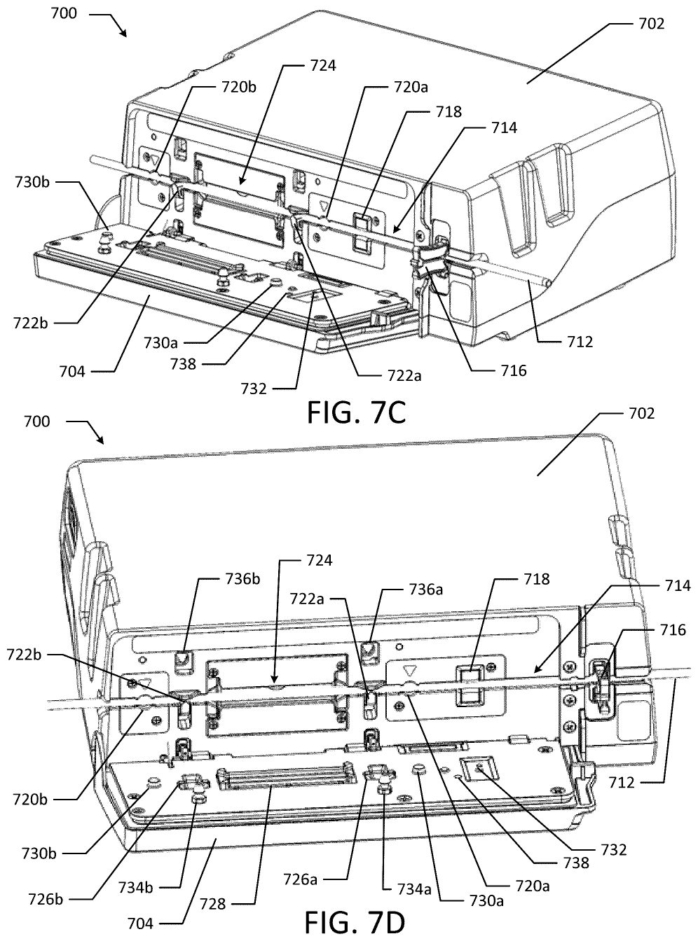

FIGS. 7A and 7B are perspective views of a peristaltic pump with a door in a closed position, according to an example embodiment of the present disclosure. FIGS. 7C and 7D are perspective views of a peristaltic pump with a door in an open position, according to an example embodiment of the present disclosure. Referring to FIGS. 7A, 7B, 7C, and 7D, a peristaltic pump 700 is used to deliver fluids (e.g., medications or nutrients) to a patient in predetermined quantities. The peristaltic pump 700 includes a housing 702, a door 704 pivotally connected to the housing 702, a display 706, and a keypad 708. The display 706 and keypad 708 are located on the door 704 along with beacon 710. The display 706 and keypad 708 are used to program the peristaltic pump 700, and more specifically, a processor in the peristaltic pump 700 to set the fluid delivery amount, etc., which is later communicated to the pumping mechanism of the peristaltic pump 700. It should be appreciated that in various other embodiments, one or more elements of the display 306 and keypad 308 could be combined in central touch screen.

The beacon 710 may be used as an indicator beacon that emits light or sound to indicate operational states or status of peristaltic pump 700. For example, when the peristaltic pump 700 is operating normally and infusing fluids, the beacon 710 may emit a solid green light. During a medium priority alarm, the beacon 710 may emit a flashing yellow light. Similarly, during a high priority alarm, the beacon 710 may emit a flashing red light. The beacon 710 may emit other combinations of colors at various intervals (e.g., pulsing, blinking, solid light) or other audible alerts to indicate the operational state or status of the peristaltic pump 700.

When the peristaltic pump 700 is in use, fluids may move through a tube loaded into the peristaltic pump 700. The tube 712 is loaded along the tube channel 714 on the peristaltic pump 700. Along the tube channel 714, the tube 712 passes through a slide clamp 716, an ultrasonic air sensor 718, an upstream pressure sensor 720a, an upstream valve 722a, the shuttle pumping region 724, a downstream valve 722b, and a downstream pressure sensor 720b. Positioned on the door 704 are other tube engagement features, such as indentions 726a, 726b and tube guide 728. The tube guide 728 is adapted to maintain the tube's position in the shuttle pumping region 724 of the peristaltic pump 700.

As illustrated in FIGS. 7C and 7D, the upstream pressure sensor 720a and the downstream pressure sensor 720b have corresponding door structures (e.g., protrusions or setscrews) that ensure the tube 712 is sufficiently held against the respective sensor. For example, protrusions 730a and 730b correspond to pressure sensors 720a, 720b. Additionally, protrusion 732 corresponds to ultrasonic air sensor 718. There may also be corresponding door indentions for each of the valves 322a, 322b. For example, indentions 726a and 726b (e.g., t-shaped indentions illustrated in FIG. 7D) are configured to prevent the tube from dislodging or “snaking” outside of the tube channel. As illustrated, the indentions 726a, 726b in door 704 are sized and shaped to prevent the tube 712 from “walking” out of valves 722a, 722b.

The door 704 may also include pegs or door latches 734a and 734b that correspond to door mounting apertures 736a and 736b in the pump housing. The door latches 734a, 734b engage with a slidable latch bar mechanism that is operatively connected to the slide clamp mechanism such that the slide clamp 716 is configured to be inserted or ejected depending on a door open or a door closed position. For example, the latch bar mechanism may be spring biased towards the downstream side of the peristaltic pump 700 (e.g., to the left when looking at FIG. 7D) and as the door 704 is closed, the door latches 734a, 734b move the latch bar mechanism to the right as the door latches 734a, 734b are pressed into the peristaltic pump housing. The door 704 may also include a magnet 738 that is associated with a Hall effect sensor in the peristaltic pump 700. The Hall effect sensor is configured to detect the presence of magnet 338 to determine whether the door 704 is closed. In an example, as a user begins to move the door 704 from an open position (illustrated in FIGS. 7C and 7D) to a closed position (illustrated in FIGS. 7A and 7B), at least one of the valves 722a, 722b may occlude the tube 712 during the closing process.

### Peristaltic Pump Sensors

The peristaltic pump 700, as previously described, has associated therewith a plurality of sensors, which are operative to provide information as to the function and location of the various elements thereof. A drive motor shaft encoder comprises an encoder flag wheel attached to the armature shaft of the motor. The peristaltic pump motor flag wheel may include a plurality of flags (e.g., twelve flags) extending radially outward from the hub thereof. These flags act in concert with optical switches to fix the location of the armature shaft of the peristaltic pump drive motor. The switches may further consist of a light emitting diode (“LED”) and a photocell. An arrangement of two optical switches allows for a first switch to sense the edge of a flag, and the second switch to sense the middle of a subsequent flag. This arrangement allows for greater resolution of motor shaft position and direction as read by the encoder. For example, the resolution of the encoder may be approximately 1/3072 of a rotation of the motor shaft.

The motor encoder senses shaft rotation directly. An index wheel may have a plurality of circumferentially coextensive radially disposed slots. Associated with these slots is an index wheel optical sensor. This sensor comprises a light emitting diode and an optical sensor or switch. In an example, the index wheel sensor is cooperative with the index wheel and the slots therein to provide positional information of the rotational location of the pump motor shaft. In operation, the index wheel sensor acts in concert with the peristaltic pump encoder to provide this positional information as well as directional information of the motor shaft. Associated with the shuttle itself is a linear gross position sensor. This sensor comprises a linear position Hall effect sensor and a plurality of magnets. Shuttle position sensor magnets present opposite poles to the shuttle Hall switch, so as to provide a field gradient operative to provide an indicium of the linear position of the shuttle.

The combination of the encoder and the other associated sensors aforementioned, provide inputs to a control mechanism, which may operate to accurately control the speed of the variable speed motor, the primary feature provided by such speed control is a temporal variability of the output of the peristaltic pump 700. Additionally, such speed control allows for an electronically controlled linearization of the pump output per individual stroke as well as improving the time-integrated output of the peristaltic pump.

The peristaltic pump 700 may also include an ultrasonic air detection apparatus or transducer. The ultrasonic transducer acts in concert with a second transducer element to detect air within the IV tubing. The peristaltic pump 700 allows the tube to be extended or stretched equally across the face of the associated sensor, thereby eliminating either a volumetric or stress gradient in the tube beneath the associated sensor so as to improve the accuracy of response of the sensor associated with, or connected to, housing. Essentially all of the sensors associated with, or actuated by, sensor arm execute the above described motion so as to achieve the above described result. The peristaltic pump may also include a downstream pressure sensor and a plurality of temperature sensors, which consist of thermistors. The slide clamp may include a Hall effect sensor to identify the presence and/or position of the slide clamp.

### Occlusion Detection

In the above introduced infusion pumps, including the syringe pump and the peristaltic pumps, occlusions may be detected by monitoring force and/or pressure measurements using various techniques. Additionally, the user may select between rapid occlusion detection and non-rapid occlusion detection. In rapid occlusion detection mode, the infusion therapy device may report an occlusion at 50% of the force or pressure thresholds discussed below.

Difference Value from Baseline

A baseline force value (e.g., a moving or sliding average window of force measurement samples, such as twenty samples) may be taken after the infusion pump motor starts. The force and/or pressure sensor may output an Analog to Digital Converter (“ADC”) count. In an example, the baseline force value may be a window of 20 samples of ADC counts after the pump motor starts. The current force measurement may be monitored and a difference value (e.g., baseline force value subtracted from the current value) may be determined. If the difference value exceeds a predetermined threshold, an occlusion alarm may sound. The infusion pump may have various settings for various occlusion detection sensitivities (e.g., Very High, High, Medium High, Medium, Low, and Very Low). In a non-limiting example, the infusion pump may generate an alarm signal when the IV line pressure reaches 2 to 4 psi for a low sensitivity alarm, 6 to 9 psi for a medium sensitivity alarm, and 14 to 16 psi for a high sensitivity alarm.

In an example, the infusion pump may generate a high priority downstream occlusion alarm for the following fluid pressures and sensitivities: (Sensitivity—Very High; Occlusion pressure 50 psi; Lower Limit 25 psi; Upper Limit 52 psi); (Sensitivity—High; Occlusion pressure 16 psi; Lower Limit 13 psi; Upper Limit 18 psi); (Sensitivity—Medium High; Occlusion pressure 13 psi; Lower Limit 10 psi; Upper Limit 15 psi); (Sensitivity—Medium; Occlusion pressure 10 psi; Lower Limit 7 psi; Upper Limit 12 psi); (Sensitivity—Low; Occlusion pressure 7 psi; Lower Limit 4 psi; Upper Limit 9 psi); and (Sensitivity—Very Low; Occlusion pressure 4 psi; Lower Limit 1 psi; Upper Limit 6 psi).

In another example, the infusion pump may generate a high priority downstream occlusion alarm for the following fluid pressures and sensitivities: (Sensitivity—Very High; Occlusion pressure 50 psi; Limit<52 psi); (Sensitivity—High; Occlusion pressure 16 psi; Lower Limit 12 psi; Upper Limit 20 psi); (Sensitivity—Medium High; Occlusion pressure 13 psi; Lower Limit 10 psi; Upper Limit 15 psi); (Sensitivity—Medium; Occlusion pressure 10 psi; Lower Limit 7 psi; Upper Limit 12 psi); (Sensitivity—Low; Occlusion pressure 7 psi; Lower Limit 4 psi; Upper Limit 9 psi); and (Sensitivity—Very Low; Occlusion pressure 4 psi; Lower Limit 2 psi; Upper Limit 8 psi).

In one example for a syringe pump, the syringe force contact is non-relaxing in nature and a change in temperature does not cause a material property change. Also, the force sensor for the syringe pump is rated and compensated to operate from −10 degrees to 40 degrees C., which covers typical syringe pump operating ranges without impacting system level temperature variations in DSO detection for the syringe pump. However, for other infusion pumps, such as a peristaltic pump, the tubing relaxes into the channel causing a change in force which is dependent on temperature. For example, the tube material properties change based on temperature and a temperature compensation slope may be added for both the baseline force value as well as current ADC values.

After the infusion pump reaches steady state, occlusion detection may be based on a change in pressure or the delta of pressure instead of the High, Medium, or Low threshold settings. For example, after reaching steady state where the pressure is very steady, a sudden shift upwards in pressure may indicate that the infusion pump is trending to an occlusion. Monitoring a delta pressure after steady state may allow for earlier occlusion detection. In an example, steady state is achieved when there is less than a 1 psi pressure change in the last two minutes of pressure measurements. If the infusion pump is not in a steady state condition, pressure delta sensing may be disabled.

The infusion pump may also monitor changes in pressure as a function of flow rate. Different baseline and/or different threshold levels may be established based on the flow rate. For example, if the difference in pressure from baseline exceeds a predetermined relationship (e.g., pressure Increase=0.3*Flowrate in a 1 minute duration), an alert or warning for an occlusion sounds.

As discussed above, a syringe force sensor or downstream occlusion (“DSO”) sensor may be located on the drive head and may be used to indicate force or pressure on the syringe plunger in a syringe pump. The calculated pressure may be used to determine a downstream occlusion. In another example, upon starting an infusion therapy, the infusion pump may record an initial baseline measurement during a first start-up interval (e.g., during the first ten seconds). At the beginning of the first start-up interval (e.g., over the first second of the ten second interval), the infusion pump may record the initial baseline measurement via a moving average of ADC counts. The baseline measurement may be updated to a lower filtered ADC value recorded within the first start-up interval. For example, if a filtered ADC value recorded at four seconds is lower than the initial baseline measurement recorded at one second, the filtered ADC value recorded at four seconds may replace the initial baseline measurement recorded at one second.

The infusion pump may continuously compute the pressure in real time as ADC values are monitored during the infusion therapy. For example, the infusion pump may continually compute the Syringe DSO Pressure according to the below equation. The Calibration Factor may be based on the syringe size and may be stored in a database that has different values for different syringe sizes.

$$Syringe DSO Pressure = \frac{( Current Filtered ADC - Current Filtered Baseline )}{Calibration Factor}$$

During the first start-up interval, occlusion detection may be based on syringe size. With syringe sizes lower than a predetermined size (e.g., 6 ml or less, 10 ml or less, etc.), occlusion detection may be determined by a dual criteria of curvature and drop. For example, an occlusion may be reported during the first start-up interval if a DSO threshold is exceeded for a threshold period (e.g., three seconds) by a plurality of filtered samples within the threshold period. Specifically, the ADC value may be monitored and a difference value (e.g., baseline value subtracted from the current ADC value) may be determined. If the difference value exceeds the DSO threshold, an occlusion alarm may sound. The quantity of samples may be predetermined and in some examples may include all filtered samples within the threshold period.

Conversely, for syringes larger than the predetermined size (e.g., 6 ml or larger, 10 ml or larger, etc.), an occlusion may be reported when a filtered sample exceeds the DSO threshold. In this example, an occlusion may be reported when any filtered sample exceeds the DSO threshold.

During a second start-up interval (e.g., during the first thirty seconds), for a rapid mode infusion therapy, an occlusion alarm may sound (when the non-rapid occlusion detection mode is selected) when the occlusion pressure threshold is exceeded. In rapid infusion therapy, the infusion pump (e.g., GUI) may calculate both the non-rapid and rapid threshold levels and report both to the infusion pump (e.g., pumphead manager). The infusion pump may select the non-rapid threshold for the second start-up interval and then may report occlusions using the rapid threshold after the second start-up interval.

After the first and second start-up intervals, a new baseline measurement may be acquired by searching for a baseline window (e.g., a five second window, ten second window, etc.) where the ADC samples within the baseline window are within a predetermined ADC value range (e.g., +/−50 ADC counts). In another example, the baseline window may be established based on a predetermined psi value range (e.g., +/−2 psi). In another example, the baseline window may be established based on a combination of a predetermined ADC value range and a predetermined psi value range, such as based on either the predetermined ADC value range or the predetermined psi value range, whichever is smaller (e.g., +/−50 ADC counts or +/−2 psi). Additionally, when establishing the baseline window, the psi value may have an upper limit (e.g., no greater than 3.87 psi above the current baseline measurement). Establishing a new baseline measurement may be performed once or multiple times for each infusion therapy start. For example, the above processes may be a one-time baseline correction per infusion therapy start. As previously mentioned, the baseline measurement may be updated to a filtered ADC value that is lower than the current established baseline.

Additionally, after the first and second start-up intervals, an occlusion may be reported when a filtered sample exceeds the DSO threshold. For example, an occlusion may be detected and reported as the threshold is crossed. As discussed above, the ADC value may be monitored and a difference value (e.g., baseline value subtracted from the current ADC value) may be determined. If the difference value exceeds the DSO threshold, an occlusion alarm may sound. Additionally, the current force measurement may be monitored by the pump and a difference value (e.g., baseline force value subtracted from the current value) may be determined. If the difference value exceeds a predetermined threshold, an occlusion alarm may sound. If rapid occlusion detection mode is selected, the syringe pump 100 may report an occlusion at 50% of the force or pressure thresholds discussed above. Rapid occlusion detection mode may be limited to lower flow rates (e.g., approximately 20 ml/hr. or less) because if the pump is performing a rapid infusion therapy with high flow rates, then occlusion detection may occur quicker as the pump is already utilizing a naturally high flow-rate.

### Slope of Pressure Measurements

An occlusion alarm may be generated if the slope calculated from the difference of two pressure measurements exceeds a threshold value. The pressure measurements may be taken in a predetermined window or time interval, for example, every two seconds. In an example, two different slope measurements may be used to account for any braking forces at the start of an infusion therapy. To prevent false alarms, the initial threshold value may be higher to account for braking forces from the tubing or other infusion pump components at start-up. After start-up, the threshold value may be lower after the pump has overcome the braking forces.

### Area Under Force Curve

Occlusion detection may also be based on energy spent or the area between a base line and the current force line. The pressure change and linear displacement (e.g., for a syringe pump) may also be used to determine whether an occlusion is present. In either of the above examples, the area calculation may be compared to a threshold value.

### Artificial Intelligence

As introduced above, the infusion pumps of the present disclosure may include an occlusion detector to determine if an infusion line is blocked. In one example, the infusion pump computes, continuously or periodically, the pressure in real time as ADC values are monitored during infusion therapy. The ADC values may be monitored by the infusion pump and used to determine a difference value. If the difference value exceeds a threshold value, an occlusion alarm is triggered. A user may select between rapid occlusion detection and non-rapid occlusion detection. In rapid occlusion detection mode, the infusion pump may report an occlusion at 50% of the force or pressure thresholds discussed below.

In another example, the infusion pump is configured to collect and store real time ADC values, which can be used to train a neural network for detecting occlusions, including upstream occlusions and downstream occlusions. Further, real time ADC values can be collected across a plurality of infusion pumps to produce a larger dataset of historical real time ADC values for training the neural network. As used herein, a neural network may refer to a computational tool capable of machine learning. The neural network may comprise a plurality of interconnected computation units known as neurons that are configured to adapt to training data, and subsequently work together to produce predictions. In some cases, the neural network may comprise, for example, a deep neural network (“DNN”), which may comprise a convolutional neural network (“CNN”). The CNN may be, for example, U-Net, ImageNet, LeNet-5, AlexNet, ZFNet, GoogleNet, VGGNet, ResNet18, or ResNet, etc.

A neural network generally includes an input layer, one or more hidden layers, and an output layer. The input layer may include nodes (e.g., X₁, X₂, . . . X_{N}) corresponding to a plurality of infusion pump parameters. In an example, three infusion pump parameters are used, which may result in a neural network having a structure with three input nodes. Each hidden layer may include a plurality of nodes for optimization (e.g., Z₁, Z₂ . . . Z_{M}). The optimization may occur by way of forward propagation, back propagation, and a calibration of weights and biases. In one embodiment, the neural network may include an output node that corresponds to an occlusion or an absence of an occlusion. For example, an output of “1” may corresponds to an occlusion while an output of “0” may correspond to no occlusion.

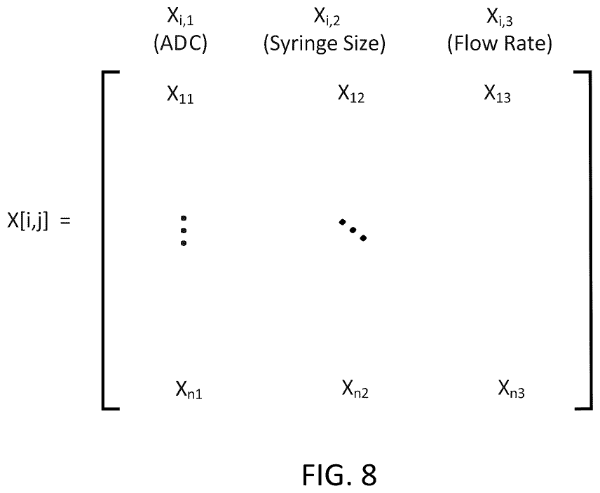

FIG. 8 is an example matrix showing an occlusion training dataset, according to an embodiment of the present disclosure. The occlusion detection training dataset, X, is a matrix that comprises N input vectors X₁, . . . , X_{N}, and can represent infusion pump parameters. As illustrated in FIG. 8, three infusion pump parameters have been chosen for input vectors X₁, . . . , X_{N} for demonstration purposes, which correspond to: (1) ADC value, (2) syringe size, and (3) flow rate. Thus, in one example, X₁₁ corresponds to an ADC value, X₁₂ corresponds to a syringe size, and X₁₃ correspond to a flow rate. Non-limiting examples of the syringe size include 1 mL, 3 mL, 5 mL, 10 mL, 20 mL, 30 mL, 50 mL, or 60 mL. Non-limiting examples of the flow rate include 0.01 mL/hr, 0.03 mL/hr, 0.05 mL/hr, 0.1 mL/hr, 100 mL/hr, 200 mL/hr, 300 mL/hr, 500 mL/hr, 650 mL/hr, or 1200 mL/hr. However, additional infusion pump parameters may be included in the occlusion training dataset. As the number of infusion pump parameters change, the dimensionality of the occlusion detection training dataset shown in FIG. 8 may change accordingly.

For example, additional infusion pump parameters may include, but are not limited to, ADC values, syringe size, flow rate, infusion fluid viscosity, type of infusion therapy, operational status of the infusion pump, length of infusion, syringe manufacturer, position of syringe pump plunger, infusion pump temperature, ambient temperature, humidity, etc. Further, the various infusion pump features introduced above may be used to collect infusion pump parameters. In one example, the infusion pump includes syringe barrel size detection via a rotary potentiometer. Syringe size as an infusion pump parameter may be collected for the occlusion detection training dataset using such syringe barrel size detection means. Additional sensor data may be used for collecting pump parameters, including a clutch sensor, a flange detection sensor, a force sensor, a syringe plunger position sensor, a syringe plunger button detect sensor, an ultrasonic air sensor, a Hall effect sensor, and a temperature sensor.

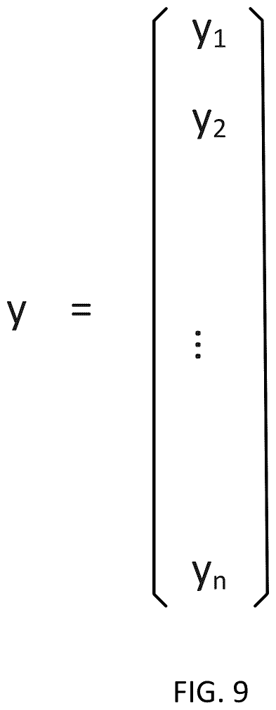

FIG. 9 is an example vector showing target values for the example occlusion training dataset of FIG. 8, according to an example embodiment of the present disclosure. In order to train the neural network, the occlusion detection training dataset may have known target values indicating whether an occlusion was present or whether an occlusion was not present. Thus, each target value in the vector shown in FIG. 9 corresponds to a three-dimensional feature vector in FIG. 8. For example, the target value of Y₁ may have a target value of “1,” indicating that an occlusion was present, and, when such occlusion was present, the infusion pump parameters corresponded to X₁₁, X₁₂, and X₁₃. In other words, the infusion pump parameters X₁₁, X₁₂, and X₁₃ in FIG. 8 correspond to infusion therapy when an occlusion was detected. Alternatively, the target value of Y₂ may have a target value of “0,” indicating that an occlusion was not present, and, when the occlusion was not present, the infusion pump parameters corresponded to X₂₁, X₂₂, and X₂₃.

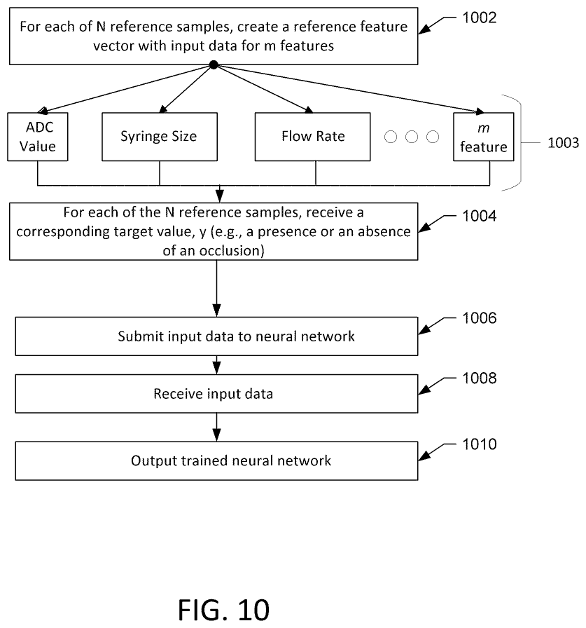

FIG. 10 is a flow chart illustrating a method for training a neural network for occlusion detection, according to an embodiment of the present disclosure. The method may be performed by one or more processors associated with an infusion pump. The training may involve an occlusion detection training dataset (e.g., as shown in FIG. 8 and FIG. 9) obtained from a plurality of reference samples. The reference samples may be collected from various infusion pump, patients, past data, databases, etc. Further, reference samples may be collected from an infusion pump over a period of time. In one example, an infusion pump operates from a first time, t₁, to a second time, t₂. From t₁ to t₂, the infusion pump parameters may be continuously collected, or collected a predetermined intervals, corresponding to no occlusion present. However, if an occlusion occurs from t₂ to a third time, t₃, the reference samples may be still be collected, but rather correspond to an occlusion present to be used in the occlusion detection training dataset.

At step 1002, the infusion pump may create, for each of a plurality of reference samples (e.g., N reference samples), a reference feature vector with input data for m features. Just as the input feature vector described in FIG. 8 was three-dimensional as a result of there being three parameters, the reference feature vector may be m-dimensional as a result of m features. As an example, the m features may include, but are not limited to, ADC value, syringe size, and flow rate, as shown in marker 1003.

At step 1004, the infusion pump may receive, for each of the N reference samples, a corresponding target value, Y, indicating either a presence or an absence of an occlusion. Just as the target values in FIG. 9 indicated either a presence or an absence of an occlusion for the occlusion detection training dataset in FIG. 8, the target values, Y, in step 1004 may indicate whether each sample received in step 1002 indicated a presence or an absence of an occlusion. The target value, Y, may comprise of N values corresponding to the N reference samples. Given the binary nature of the indication (e.g., a presence or an absence of an occlusion), the target value may comprise, for example, “0” to indicate the absence of an occlusion or “1” to indicate the presence of an occlusion.

At step 1006, the infusion pump uses various inputs to train the neural network. In one embodiment, the infusion pump inputs a reference feature vector with input data for m features with a corresponding target value, Y, indicating either a presence or an absence of an occlusion into the neural network. As previously introduced, the inputs to train the neural network include, for example, the occlusion training dataset of FIG. 8 and a corresponding example vector showing a target value. Thus, the inputs used to train the neural network represent various infusion parameters that are correlated to known occlusions or conditions when occlusions are not present.

At step 1008, the neural network receives the inputs for training and optimizing an algorithm. As a result of training the neural network, the processing output at step 1010 is a trained neural network. In one example, the trained neural network is a discrete mathematical formula of weighted values, window size, and window overlap. In an example, the processing output of the trained neural network is a Boolean determination of an occlusion flag, corresponding to 1, or no-occlusion, corresponding to 0. The trained neural network can then be applied to an infusion pump to detect occlusions in real time.

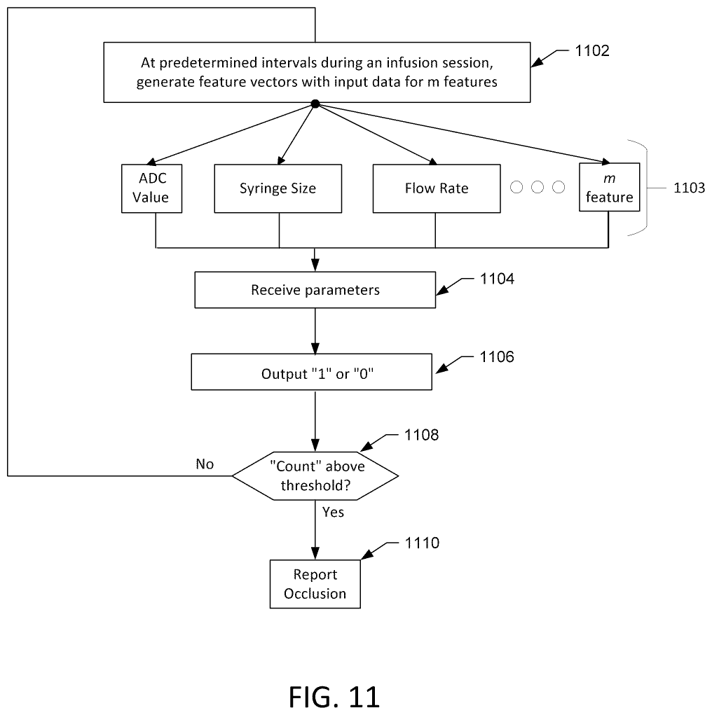

FIG. 11 is a flow chart illustrating a method for occlusion detection in infusion pumps using a trained neural network, according to an embodiment of the present disclosure. At step 1102, the infusion pump may generate, at predetermined intervals during an infusion therapy session, feature vectors with input data for m features. The m features may include, for example, ADC value, syringe size, and flow rate as shown in marker 1103. The trained neural network receives the input data at step 1104 and outputs a “1” or “0” at step 1106, which corresponds to an occlusion flag or no occlusion flag.

At step 1108, an algorithm receives the output from step 1006, keeping a count of occlusion flags. If a “1” is output at step 1006, the count increases by “1” in step 1008. In step 1108, the infusion pump determines whether the count of occlusion flags is above a predetermined threshold. If the count is above a predetermined threshold, an occlusion is reported at step 1110. If the count is below the predetermined threshold, steps 1102 to 1108 are repeated. In an example, the predetermined threshold is 40 occlusion flags.

As shown in FIG. 11, the example three-dimensional dataset may be used to detect occlusion in real time. However, the trained neural network is also capable of detecting occlusion only based on real time ADC values. In other words, a one-dimensional dataset consisting only of real time ADC values may be used as an input to the trained neural network to detect occlusion. Based on the previous training process, the trained neural network is configured to derive additional parameters from the ADC value, which can be used for occlusion detection.

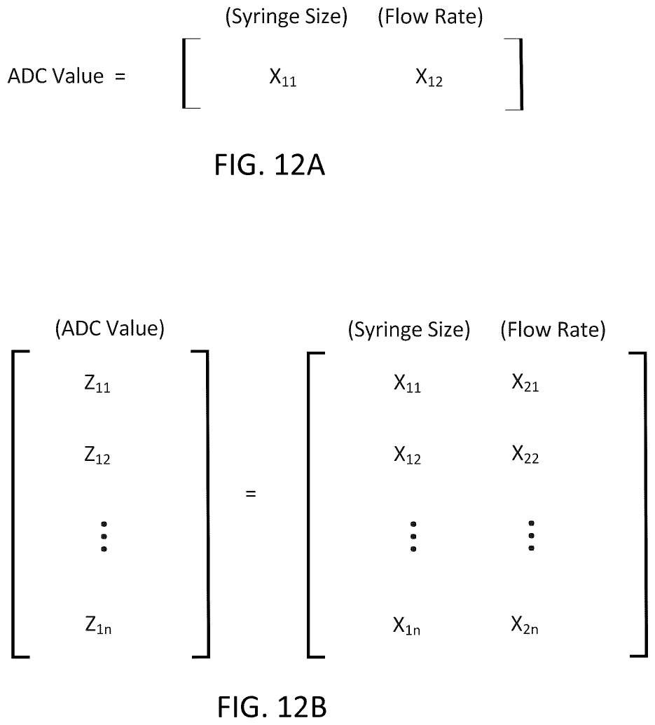

FIGS. 12A and 12B are example vectors showing an ADC value that corresponds to infusion pump parameters, according to an embodiment of the present disclosure. As illustrated in FIG. 12A, an ADC corresponds to a syringe size and a flow rate. Thus, in one example, the infusion pump may input an ADC value into the trained neural network, which can derive additional infusion pump parameters for occlusion detection. Similarly, FIG. 12B shows several ADC values that correspond to a matrix of infusion pump parameters. Therefore, the infusion pump at step 1102 of FIG. 11 may generate feature vectors that are input into the trained neural network. By relying on the trained neural network to derive additional infusion pump parameters from the ADC value, the infusion pump is not required to collect these additional infusion pump parameters.

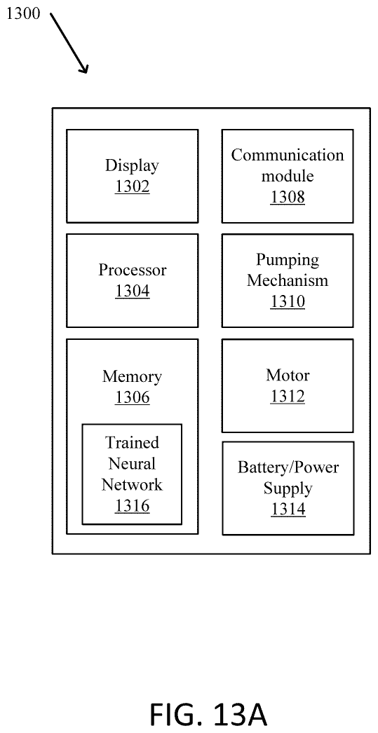

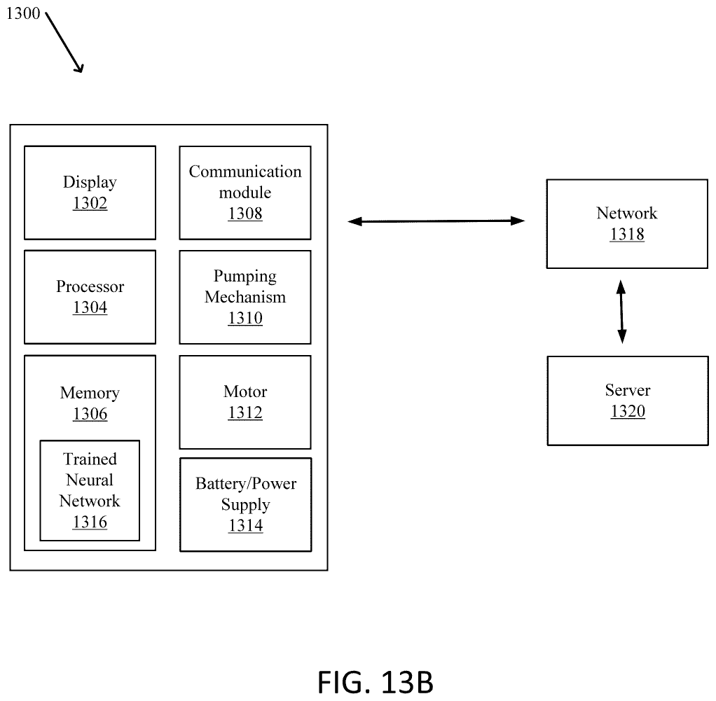

FIGS. 13A and 13B are high-level component diagrams of an infusion pump, according to an example embodiment of the present disclosure. The infusion pump 1300 is an infusion therapy pump, such as a syringe pump as illustrated in FIG. 1 or a peristaltic pump as illustrated in FIG. 7. The infusion pump 1300 includes a processor 1304 in communication with memory 1306, which is powered by a battery or power supply 1314. The processor 1304 communicates with a display 1302, a motor 1312 and associated pumping mechanism 1310, and a communication module 1308.

The power supply 1314 may take many different forms. In one preferred embodiment, the power supply 1314 may be in the form of a rechargeable battery unit. Additionally, the pump may be powered from an AC power supply. The AC power supply assembly has a power cord and an associated terminal that plugs into the housing. The AC power supply assembly has a plug that is configured to be inserted into a standard electrical outlet to recharge the rechargeable battery when necessary. The AC power can also be supplied through the assembly to power the pump.

As introduced above, the infusion pump 1300 includes a processor 1304. While one processor 1304 is shown, the infusion pump 1300 may include a plurality of processors. The processor 1304 includes a controller, a logic device, etc. configured to execute the trained neural network 1316 (e.g., an algorithm) stored in the memory 1306. The processor 1304 is also configured to execute one or more instructions stored in the memory 1306 that, when executed by the processor 1304, cause the processor 1304 to perform the operations described herein to provide an infusion therapy treatment. The memory 1306 includes any memory device including read only memory, flash memory, random access memory, a hard disk drive, a solid-state drive, etc.

In some embodiments, the processor 1304 is configured to perform the method described in connection with FIG. 11. In these embodiments, the processor 1304 receives real time ADC values from the infusion pump, which is input into the trained neural network 1316. The processor 1304 then receives an output from the trained neural network 1316 to indicate whether an occlusion is detected. When an occlusion is detected, the processor 1304 may cause an alert and/or an alarm to be displayed on the display 1302. The processor 1304 may perform the method described in connection with FIG. 11 continuously, or at periodic intervals, such as every 50 milliseconds, 100 milliseconds, 500 milliseconds, 1 second, 2 seconds, etc.

The communication module 1308 is configured for wireless and/or wired communication with a network, such as the Internet, a cellular network, and/or a local hospital network. The communication module 1308 may be configured, for example, for Wi-Fi or Ethernet communication. In the illustrated example, the communication module 1308 is configured to receive the trained neural network 1316 from a server or clinician computer via a network. In other examples, the processor 1304 may perform the method of described above to train the neural network 1316. The communication module 1308 may also receive one or more parameters specifying an infusion therapy treatment to be performed. Further, the communication module 1308 may transmit alert or alarm messages to a server when an occlusion is detected.

FIG. 13B is a diagram of an infusion pump 1301, according to an example embodiment of the present disclosure. The infusion therapy system 1301 includes the infusion therapy device 1300 of FIG. 13A. The infusion therapy system 1301 includes a server 818 that is connected to the infusion therapy device 1301 via a network 1318, which may include any cellular, wide area, and/or local area network. The server 1320 may be part of a heath information system and include a clinician computer.

In the illustrated example, the server 1320 receives reference training data. The training data may be input into the server 1320 from manually obtained data. Additionally or alternatively, the training data may be received from one or more infusion therapy devices including the infusion pump 1300. As discussed above, the server 820 is configured to create one or more trained neural networks 816 for occlusion detection using the historical ADC values. The server 1320 may transmit the trained neural networks to the infusion therapy device 1300 via the network 1318.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

1. An infusion pump for detecting an occlusion, the infusion pump comprising:
a pumping mechanism operable with a portion of intravenous (“IV”) tubing for providing controlled delivery of a fluid from a container to a patient;
one or more processors; and
a memory storing instructions that, when executed by the one or more processors, cause the one or more processors to:
input data at predetermined intervals during an infusion session into a trained neural network, wherein the trained neural network outputs an occlusion flag or a non-occlusion flag; and
generate an alert when the trained neural network outputs an amount of occlusion flags above a predetermined threshold.

2. The infusion pump of claim 1, wherein the trained neural network comprises an input layer, a hidden layer, and an output layer.

3. The infusion pump of claim 2, wherein the output layer of the trained neural network outputs the occlusion flag or the non-occlusion flag.

4. The infusion pump of claim 1, wherein the data is a three-dimensional dataset comprising a plurality of input vectors, wherein each one of the plurality of input vectors corresponds to a plurality of infusion pump parameters.

5. The infusion pump of claim 4, wherein the plurality of infusion pump parameters comprises an ADC value, a flow rate, and a syringe size.

6. The infusion pump of claim 1, wherein the data is an ADC value.

7. The infusion pump of claim 6, wherein the trained neural network derives a plurality of infusion pump parameters from the ADC value.

8. The infusion pump of claim 1, wherein the predetermined threshold is forty occlusion flags.

9. The infusion pump of claim 1, wherein the trained neural network comprises a Convolutional Neural Network (“CNN”).

10. The infusion pump of claim 9, wherein the CNN comprises a Residual Network (“ResNet”) Architecture.

11. The infusion pump of claim 1, wherein the one or more processors are configured to cause an alert or an alarm to be displayed on a user interface when the amount of occlusion flags exceed the predetermined threshold.

12. The infusion pump of claim 1, wherein the one or more processors are configured to pause or terminate the infusion session when the amount of occlusion flags exceed the predetermined threshold.

13. A method of training a neural network for detecting an occlusion in an infusion pump, the method comprising:
collecting a plurality of infusion pump parameters;
correlating the plurality of infusion pump parameters to an occlusion state of the infusion pump, wherein the occlusion state corresponds to a time at which the plurality of infusion pump parameters was collected; and
inputting the plurality of infusion pump parameters correlated to the occlusion state into the neural network.

14. The method of training a neural network for detecting an occlusion in an infusion pump of claim 13, wherein the plurality of infusion pump parameters is collected from a plurality of infusion pumps.

15. The method of training a neural network for detecting an occlusion in an infusion pump of claim 13, wherein the occlusion state represents an occlusion or no occlusion.

16. The method of training a neural network for detecting an occlusion in an infusion pump of claim 13, wherein the neural network comprises a Convolutional Neural Network (“CNN”).

17. The method of training a neural network for detecting an occlusion in an infusion pump of claim 16, wherein the CNN comprises a Residual Network (“ResNet”) Architecture.

18. An infusion pump method for detecting an occlusion, the method comprising:
determining a real time ADC value;
applying the real time ADC value to a trained neural network, wherein the trained neural network outputs a Boolean determination corresponding to an occlusion flag; and
generating an occlusion alarm when a number of occlusion flags exceed a threshold.

19. The infusion pump method for detecting an occlusion of claim 18, wherein the trained neural network derives a plurality of infusion pump parameters from the real time ADC value.

20. The infusion pump method for detecting an occlusion of claim 18, wherein one or more processors are configured to cause an alert or an alarm to be displayed on a user interface when the amount of occlusion flags exceed a predetermined threshold.
